(19) United States
(12) Patent Application Publication
(10) Pub. No.: US 2014/0298496 A1
(43) Pub. Date: Oct. 2, 2014

(54) PHENOCOPY MODEL OF DISEASE

(71) Applicants: Adrian Krainer, Huntington Station, NY (US); Kentaro Sahashi, Huntington, NY (US)

(72) Inventors: Adrian Krainer, Huntington Station, NY (US); Kentaro Sahashi, Huntington, NY (US)

(73) Assignee: Cold Spring Harbor Laboratory, Cold Spring Harbor, NY (US)

(21) Appl. No.: 14/128,446

(22) PCT Filed: Jun. 22, 2012
(86) PCT No.: PCT/US12/43894

§ 371 Date: May 27, 2014

Related U.S. Application Data
(60) Provisional application No. 61/500,597, filed on Jun. 23, 2011.

(51) Int. Cl. A01K 67/027; C12N 15/113

(52) U.S. Cl. CPC A01K 67/0275; C12N 15/113
USPC 800/9

(57) ABSTRACT

Methods and compositions for generating nonhuman disease models through splicing modulation.

### RELATED APPLICATION

This application claims the benefit of the filing date of U.S. Provisional application 61/500,597, filed Jun. 23, 2011. The entire teachings of the referenced application are incorporated herein by reference.

### BACKGROUND

Spinal muscular atrophy (SMA) is characterized by skeletal-muscle weakness due to progressive loss of spinal α-motor neurons. A decrease in SMN protein, which regulates snRNP assembly, is responsible for SMA, but it is not understood how this causes motor-neuron degeneration (Burghes, A. H. & Beattie, C. E., Nat Rev Neurosci 10, 597-609 (2009)). Humans have a closely related SMN2 gene (hSMN2) that predominantly expresses a defective truncated protein, due to alternative splicing of exon 7 (Lorson, C. L. et al. Nat Genet 19, 63-66 (1998)). SMN2 acts as a disease modifier, reducing disease severity with increased copy number (McAndrew, P. E. et al. Am J Hum Genet 60, 1411-1422 (1997)).

### SUMMARY

Described herein are methods and compositions for generating nonhuman animal disease models through alternative splicing of mRNA, including any type of alternative splicing that results in production of isoform(s) that cause or contribute to a disease phenotype, particularly a human disease phenotype. Methods described herein make use of splicing modulation, such as exon-skipping, to produce non-human animal models of mis-splicing associated diseases, such as those in which alternative splicing results in skipping or failure to incorporate an exon or an exon portion needed to produce the functional protein otherwise produced (produced in the absence of mis-splicing in the non-diseased state). Described herein are antisense olignucleotides (ASOs) that are designed to cause sustained splicing defects, such as antisense oligonucleotides that specifically alter splicing patterns of target mRNAs and modulate gene splicing, particularly ASOs that exacerbate or cause mis-splicing. Also described is use of the ASOs to produce nonhuman animal models that phenocopy a genetic disease, such as a genetic disease caused by altered splicing of a gene, with the result that there is abnormal expression of the encoded protein (e.g., reduced, such as suboptimal, levels of functional protein; expression of protein with reduced functionality or both). Such splicing modulating ASOs are also referred to herein as synthetic exon-skipping antisense oligonucleotides. The splicing modulating ASOs and methods described herein have been used to induce pathogenesis and accurately model splicing-associated disease in nonhuman animals.

Methods described herein are useful to produce a nonhuman animal that phenocopies a disease, such as a genetic disease, neurodegenerative disease, cancer, psychiatric disorder (e.g., schizophrenia, autism, frontotemporal dementia), metabolic disorder, cardiovascular disorder, or premature aging disorder, that results from a splicing defect. The present method and splicing modulating ASOs described herein have broad applicability. That this is the case is evidenced, for example, by the frequent occurrence of splicing mutations associated with human diseases and the availability of extensive information about mutations associated with human inherited diseases. For example, it has been estimated that about “one third of all disease-causing mutations alter pre-mRNA splicing.” (Lim, K. H. et al. Proc Natl Acad Sci 108, 11093-11098 (2011)). The Human Gene Mutation Database (hgmd.cf.ac.uk), which is a collection of “data on germ-line mutations in nuclear genes underlying or associated with human inherited disease,” provides information for 11,525 mutations noted as having “consequences for mRNA splicing” and specifies “the relative position of the lesion with respect to a numbered intron donor or acceptor splice site.” In addition, there are methods available for identifying splicing elements and predicting pre-mRNA processing defects, such as Spliceman, an online tool for predicting pre-mRNA processing defects in human genes, described by Lim et al. (Lim, K. H. et al. Proc Natl Acad Sci 108, 11093-11098 (2011) and at fairbrother.biomed.brown.edu/spliceman).

In one embodiment, the method is a method of producing a nonhuman animal that phenocopies symptoms of a genetic disease caused by altered splicing of a gene, referred to as the target (disease) gene. The method comprises administering to a nonhuman animal, such as a rodent (e.g., rat, mouse), which has been modified (e.g., by producing a transgenic animal) to comprise the human disease gene (e.g. a gene that is mis-spliced in the disease being phenocopied and causes the disease in humans), splicing modulating synthetic antisense oligonucleotides (ASOs) that cause or exacerbate mis-splicing of the target gene, thereby producing a nonhuman animal that comprises splicing modulating synthetic ASOs and phenocopies symptoms of the disease. In an alternative embodiment, the nonhuman animal has been modified (e.g., by producing a transgenic animal) to comprise a human wildtype (normal) gene, such as a human wildtype gene that exhibits alternative splicing, and ASO that change the alternative splicing of the normal gene are administered in an amount effective to alter splicing of the wildtype gene. As a result of the change in alternative splicing, different isoform(s) can be produced and/or isoforms normally expressed can be expressed at levels different from the expected level for each isoform (e.g., higher or lower levels of isoforms normally expressed). In certain embodiments, a nonhuman gene that is sufficiently similar to the human gene to be a useful proxy is used to produce the transgenic nonhuman animal. The nonhuman gene can be sufficiently similar to a human disease gene (e.g., one that exhibits mis-splicing) or sufficiently similar to a wildtype human gene that exhibits alternative splicing. The method can be applied to any disease caused by alternative splicing of RNA, such as exon mis-splicing.

In some of the embodiments, an animal used in the method comprises its ortholog of a normal (wildtype) human gene, such as a human gene that exhibits alternative splicing or comprises its ortholog of the human disease gene and does not need to modified to include the human gene being assessed. ASOs that specifically target the normal gene can be administered to alter splicing of the wildtype gene, as described above for human wildtype genes and ASOs that target the corresponding mutation in the animal ortholog of the human disease gene can be administered, also as described above. In one embodiment, instead of making a transgenic animal (rodent, monkey, pig, etc) one would administer an ASO that targets the normal animal ortholog of the human gene, causing it to missplice. Products of the mis-splicing will be, for example, products that are not normally produced from the normal animal ortholog and/or products that are produced at different levels (higher or lower) than would be produced in the absence of the ASOs. In one embodiment, the normal mouse Smn gene is targeted and skipping of exon 7 is promoted.

In one embodiment, the synthetic ASOs are complementary to a site on pre-mRNA of the target gene, the nonhuman animal is a nonhuman neonate, such as a transgenic nonhuman neonate, and splicing modulating ASOs are administered by intracerebroventricular (ICV) injection, intrathecal administration or systemically, such as subcutaneously, intraperitoneally, intravenously or intramuscularly. The ASOs can be 2′-O-(2-methoxyethyl) (MOE) ASOs or other chemically modified ASOs, such as morpholino or LNA modified ASOs.

A further embodiment is a method of producing a nonhuman animal, such as a rodent, (e.g., a mouse, rat) model that phenocopies a neurodegenerative disease, such as a human neurodegenerative disease, in which the nonhuman animal is a transgenic animal (mouse, rat) that lacks an endogenous gene that is essential to the nonhuman animal (e.g., SMN1 gene) and has been modified to include a corresponding/similar human gene that is mis-spliced in humans and rescues the nonhuman animal from embryonic lethality (e.g., human SMN2 gene). In the method, synthetic ASOs that specifically alter the splicing pattern of the corresponding human gene (e.g., alter the splicing pattern of human SMN2) and exacerbate the splicing defect (mis-splicing) are administered, thereby producing a nonhuman animal in which the target gene (e.g., hSMN2) is mis-spliced more frequently than would otherwise occur and which phenocopies (exhibits characteristics of) the human neurodegenerative (or other) disease. In a specific embodiment, the method is carried out, as described above with regard to producing a nonhuman animal model, in any animal in which a gene equivalent to SMN1 is essential and has been deleted or rendered nonfunctional and human SMN2 has been introduced and is expressed.

A specific embodiment of the method is a method of producing a non-human animal that phenocopies a human disease, caused by altered splicing of a human gene, referred to as a human disease gene, comprising administering splicing modulating synthetic ASOs that initiate mis-splicing of the human disease gene to a non-human animal, wherein the animal comprises: (a) the wildtype of the human disease gene or (b) an endogenous gene corresponding to (an ortoholog of) the wildtype of the human disease gene, wherein the synthetic ASOs are administered in sufficient quantity to produce an animal that phenocopies the human disease. In specific embodiments, the human disease that is phenocopied is a genetic disease, neurodegenerative disease, cancer, psychiatric disorder (e.g., schizophrenia, autism, frontotemporal dementia), metabolic disorder, cardiovascular disorder, or premature aging disorder, that results from a splicing defect.

ASOs can be produced using known methods and their synthesis makes use of the Watson-Crick base pairing: G pairs with C; A pairs with U (or T). U can also pair with G (wobble base pairing). ASOs can be of widely varying length. The appropriate length of ASOs needed can be determined empirically with reference to the target site and can be any length that permits them to function in the subject methods (e.g., from about 5 to about 100 nucleotides, any length from about 5 to about 20 nucleotides or any length from about 15 to about 20 (e.g., 15, 16, 17, 18, 19, 20) nucleotides. Typical ASOs are 15-20 nucleotides. Backbone and base modifications of ASOs can be of a wide variety of types and ASO should be sufficiently modified to avoid RNase H cleavage of the target pre-mRNA. Other modifications that can be made stabilize the ASO, promote its cellular uptake, increase the stability of binding to the target, etc.

The sequence of the ASO dictates precisely which target RNA it binds, as well as where along the target RNA it binds, according to well-known base pairing rules. Repetitive sequences are avoided in ASOs, so that ideally the ASO binds only (or mostly) to one location in the transcriptome. This can be predicted by searching against the entire genome or transcriptome sequence. ASO can be designed to bind a known element involved in splicing, such as a splice site, splicing enhancer, splicing silencer, in an intron or exon, or overlapping region. Or it can disrupt or promote higher-order structure in the pre-mRNA in a way that can affect the resulting splicing pattern. Another approach is to systematically search for effective ASOs by individually testing ASOs complementary to overlapping segments along the target pre-mRNA, or a region of interest within it. See, for example Hua, Y. et al. Am J Hum Genet 82, 834-848 (2008) for description of how ASOs can be designed/identified.

As described in detail herein, specifically designed ASOs have been used to accurately model a splicing-associated disease, as exemplified by SMA, in nonhuman animals. SMA is a motor-neuron disease caused by loss-of-function mutations in the SMN1 gene. The related human SMN2 gene expresses suboptimal levels of functional SMN protein, due to alternative splicing that skips exon 7. As described in detail herein, a nonhuman animal model of a neurodegenerative disease (SMA) has been produced through the use of splicing modulating ASOs. The nonhuman animal model phenocopies symptoms of the human neurodegenerative disease. In specific embodiments, exon-skipping synthetic ASOs are specific for SMN2 and inhibit (partially or completely) normal splicing. In particular embodiments, the splicing modulating ASOs target a site on exon 7 of SMN pre-mRNA and, as a result, inhibit exon 7 splicing and promote pathogenesis. In a specific embodiment, the site on SMN2 pre-mRNA that is targeted by the splicing modulating ASO comprises: AAGAAGGAAGGTGCTCAC (SEQ ID NO.: 13) and ASOs comprise any nucleic acid residues sufficiently complementary, in whole or to part, to SEQ ID NO.: 13 that they hybridize to the site on SMN2 pre-mRNA under the conditions used (such as those in cultured cells or patient cells are maintained for assessment or conditions in nonhuman animal (e.g., mouse) cells or tissues, either removed from the nonhuman animal or in an intact nonhuman animal).

In specific embodiments, splicing modulating ASOs comprise the sequence of ASO-20-37 (5′-GTGAGCACCTTCCTTCTT-3′; SEQ ID NO.: 10), the sequence of ASO-23-42 (5′-GGAATGTGAGCACCTTCCTT-3′; SEQ ID NO.: 11), an ASO having a sequence sufficiently similar to that of SEQ ID NO.: 10 that the ASO hybridizes to sequences, such as a site on SMN2 pre-mRNA, to which SEQ ID No.: 10 hybridizes, or an ASO having a sequence sufficiently similar to that of SEQ ID NO.: 11 that the ASO hybridizes to sequences, such as a site on SMN2 pre-mRNA, to which SEQ ID NO.: 11 hybridizes. An ASO hybridizes to its target pre-mRNA in the nucleus of a cell under physiological conditions, after it is taken up by the cell.

RNA-targeted methods for generating animal disease models have largely focused on RNAi-based or antisense-knockdown approaches (Sandy et al. 2005; Crooke 2007). Unlike these approaches, the method described herein, referred to as TSUNAMI (for targeting-splicing using negative ASOs to model illness), retains the primary transcript, allowing the testing of therapeutics that correct mis-splicing. Splicing-modulating ASOs do not cause RNA cleavage, and thus have fewer potential off-target hybridization effects than knockdown ASOs or RNAi. Furthermore, ICV-injected MOE ASO at the effective doses induces minimal or no chemistry-related neuroinflammation, as demonstrated by the limited extent of Aif1 mRNA expression. Indeed, the ASOs described herein did not affect normal or heterozygote Smn^{+/-} SMN2^{+/+} mice—reflecting their species-specific effects on splicing—ruling out off-target effects. In addition, two different ASOs targeting SMN2 exon 7 elicited the same SMA-like phenotype, again ruling out off-target effects. Moreover, the phenotypic amelioration of ASO-20-37-treated mice with therapeutic ASO-10-27 definitively proved that the SMA-like phenotypes were induced through SMN2 splicing inhibition.

Although genetic strategies are also very useful for temporal and spatial regulation of SMN expression in mice (Park et al. 2010b; Le et al. 2011; Lutz et al. 2011), TSUNAMI has several advantages: i) it circumvents the need to engineer mouse strains, which requires expertise and time-consuming genetic crosses and characterization of individual lines; ii) it provides great flexibility in fine-tuning disease severity, simply by changing the ASO dose or choosing ASOs with different potencies; iii) it can help define the relevant tissues and cell types in a given disease by taking advantage of ASO pharmacokinetic properties, which depend on the administration route, chemical modifications, and formulation; iv) it facilitates the temporal regulation of target gene expression, simply by delivering the ASO at various prenatal or postnatal stages, varying the dosing frequency, or using different chemical modifications to adjust the ASO half-life; and v) it enables the testing of splicing-correcting therapeutics to prevent, delay, or rescue the disease phenotype.

### BRIEF DESCRIPTION OF THE DRAWING

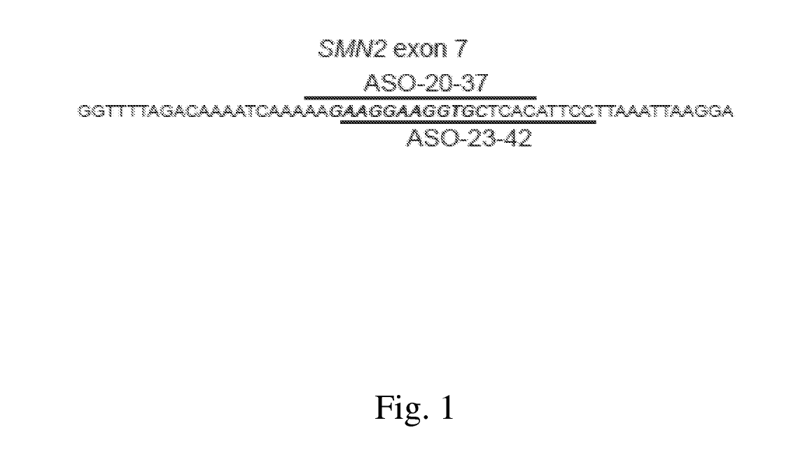

The FIG. 1 shows SMN2 exon 7 sequence and a schematic representation of ASO-20-37 and ASO-23-42.

### DETAILED DESCRIPTION

RNA splicing requires recognition of pre-mRNA cis-acting elements by spliceosome components and auxiliary RNA-binding proteins (Cartegni, L., Chew, S. L. & Krainer, A. R. Nat Rev Genet 3, 285-298 (2002)). Synthetic ASOs can be designed to base-pair with high specificity to a cis-element on a given pre-mRNA, so as to hinder binding of splicing activators or repressors, thereby altering splicing patterns. Based on these properties, ASOs are being developed as RNA-targeted therapeutics to correct disease-associated splicing defects or—in the case of Duchenne muscular dystrophy—to skip exons and suppress frame-disrupting mutations (Aartsma-Rus, A. RNA Biol 7, 453-461 (2010)).

Exon-skipping ASOs are being used therapeutically, notably to restore the translational reading frame in Duchenne muscular dystrophy (DMD) (Aartsma-Rus 2010). In contrast, methods described herein make use of them to generate a disease model.

Diseases for which a nonhuman animal model can be produced using exon-skipping synthetic ASOs as described herein include neurodegenerative diseases, such as SMA and familial dysautonomia. The animal model can be a model produced by introducing ASOs that increase (initiate, cause or exacerbate) altered splicing, such as mis-splicing of an exon, into a normal animal (an animal that does not have the disease to be phenocopied) or by introducing ASOs that cause or exacerbate mis-splicing of an exon into a transgenic animal (e.g., a transgenic rodent, such as a transgenic mouse). The transgenic animal can comprise, for example, a normal human gene to be targeted by ASOs or a disease associated gene; in the latter instance, the transgenic nonhuman animal can exhibit at least some of the symptoms of the disease to be phenocopied, such as a transgenic rodent that exhibits mild-moderate symptoms of SMA. In embodiments in which the transgenic rodent, such as a transgenic mouse, exhibits symptoms of SMA, the transgenic animal can be one that lacks Smn, such as Type III SMA mice (Smn^{-/-}; SMN2^{+/+}), such as those available from The Jackson Laboratory, or Type I SMA mice generated by crossing (Smn^{-/-}; SMN2^{+/0}). Other transgenic animals from which the nonhuman animal model can be produced include other rodents (e.g. a rabbit, a squirrel), a primate, a pig, a sheep, or other animal that has a gene corresponding to the mouse Smn gene (e.g., a gene that is essential to the animal and whose absence results in embryonic lethality, which can be rescued by expression of hSMN2. Splicing modulating ASOs, animal models and their uses described herein are useful for phenocopying diseases, including neurodegenerative diseases, caused by altered splicing of a gene (e.g., SMA, familial dysautonomia) and identifying potential therapies (e.g., splicing modulating ASOs, therapeutic proteins, small molecules, gene therapy). In specific embodiments, the model is a mouse model of intermediate SMA or of familial dysautonomia.

Another example of a genetic disease in which alternative splicing is the cause and for which the antisense method of phenocopying is useful to produce a non-human animal model is familial dysautonomia (Riley-Day syndrome). It is caused by a 5′ splice site mutation in intron 20 of IKBKAP, which causes skipping of exon 20 (work by S. Slaugenhaupt and others). Cartegni et al describe missense alleles and silent alleles that affect splicing of various disease genes. Both types of alleles and, particularly silent alleles, are of interest in identifying ASOs useful for producing a nonhuman animal model that phenocopies familial dysautonomia. Using methods described herein and by Hua, Y. et al. Am J Hum Genet 82, 834-848 (2008), ASOs can be designed, such as by testing ASOs in the introns flanking intron 20 or the exon itself, to find one that promotes exon inclusion. Such mutations cause exon skipping. The resulting ASOs are assessed for their function and use in causing or enhancing altered splicing by, for example, RT-PCR in patient fibroblasts. ASOs that target specifically (are complementary to) a mutation in IKBKAP pre-mRNA (e.g., a 5′ splice site mutation in intron 20) are splicing modulating ASOs that can be used to cause sustained splicing defects in nonhuman animals and phenocopy familial dysautonomia.

In the methods described herein, splicing modulating ASOs can be administered by a variety of routes and the appropriate route(s) can be determined empirically, taking into consideration, for example, the type and size of the nonhuman animal and the location(s) to which splicing modulating ASOs are to be delivered. Splicing modulating ASOs can be administered, for example, by intracerebroventricular (ICV) injection, by intrathecal administration (by infusion or bolus) or systemically (e.g., subcutaneously, intraperitoneally, intravenously, intramuscularly).

ASOs can be of any length that permits them to function in the subject methods and will generally be any length between from about 5 to about 100 nucleotides, any length from about 5 to about 20 nucleotides or any length from about 15 to about 20 (e.g., 15, 16, 17, 18, 19, 20) nucleotides long. Splicing modulating ASOs used in the methods described herein can be modified or unmodified. A wide variety of modifications known to those of skill in the art can be made. They include, but are not limited to, use of 5-methylcytosines to modify some or all of the cytosine bases (e.g., 5-Me-C can be used to reduce inflammatory effects of some sequences), use of a phosphorothioate backbone and other modifications, such as morpholino, 2′-O-methyl, 2′-F, LNA and 2′-O-(2-methoxyethyl). The dose and frequency of administration of splicing modulating ASOs appropriate for producing a particular nonhuman animal model vary, depending on such considerations as the disease whose phenocopy is sought and the animal used. In some embodiments, splicing modulating ASOs will be administered to neonatal animals (e.g., as described herein for one embodiment of producing a nonhuman model of intermediate “type II” SMA). In others, they will be administered to older animals, including pre-adult and adult animals (e.g., to determine whether normal levels of protein, such as normal levels of full-length SMN, are needed after early development).

In a specific embodiment, the disease that is phenocopied in the nonhuman animal model is SMA, including severe “type I” SMA, intermediate “type II” SMA and mild “type III” SMA. The ability to model each of the three main types (or other subtypes) of SMA is useful. Particularly useful are intermediate “type II” SMA models because currently-available SMA mouse strains fall short of providing phenotypically accurate models for the intermediate “type II” disease. Nonhuman models described herein can be used to analyze phenotype, pathophysiology and therapeutic efficacy of candidate treatments.

In one embodiment, the nonhuman animal model is an animal that, as the wild type (normal) animal has at least one copy of a gene, such as SMN1, that is equivalent to a human gene (e.g., human SMN1) essential to the nonhuman animal and whose absence is lethal. In the nonhuman animal model, the essential gene is lacking (either physically or functionally). The model comprises a human gene (or equivalent nonhuman gene) that is mis-spliced in humans with a disease of interest (e.g., SMA) and rescues the non-human animal from lethality caused by lack of the essential gene. In these embodiments, synthetic exon-skipping ASOs that inhibit SMN2 splicing in the nonhuman animal are administered via a route that results in synthetic exon-skipping ASOs circulating in cerebral spinal fluid (CSF) and being distributed throughout the central nervous system (CNS). For example, the synthetic exon-skipping ASOs are introduced through one or more intracerebroventricular injections, which delivers them to the brainstem and spinal cord, the loci of α-motor neurons. The dose size and number of doses administered by ICV injection needed to modulate exon 7 splicing so as to cause its mis-splicing and produce a nonhuman animal that phenocopies SMA can be determined empirically using, for example, the synthetic exon-skipping ASOs described herein (e.g., those identified as SEQ ID NO.: 10, those identified as SEQ ID NO.: 11 and any that are sufficiently similar in sequence to either the sequence of SEQ ID NO. 10 or the sequence of SEQ ID NO. 11 that they hybridize in substantially the same way as SEQ ID NO. 10 or SEQ ID NO. 11 to a target, such as a site on exon 7 pre-mRNA). The splicing modulating ASOs can be administered at a variety of points in the life of the nonhuman animal and the time at which administration will be done (e.g., neonatally, in young/preadult animals, adult animals) is determined with reference to the desired effect (e.g., the desired phenocopy). For example, neonatal ICV injection has been shown to phenocopy SMA in mice, as described herein.

As described herein, in one embodiment, a single intracerebroventricular splicing modulating ASO injection in neonatal mice recapitulates SMA-like progressive motor dysfunction, growth impairment, and shortened life span in a dose-dependent manner. These phenotypes are rescued by a therapeutic ASO that restores correct splicing. The work described uncovered starvation-induced splicing changes, particularly in SMN2, which may accelerate disease progression in severe SMA. Introducing splicing modulating ASOs that cause sustained splicing defects, as described herein, is useful as a general strategy to induce pathogenesis and model a variety of diseases in nonhuman animals.

Described herein are methods of producing a nonhuman animal that phenocopies a genetic disease, such as a neurodegenerative disease, such as a motor-neuron disease, that results from a splicing defect (which, in turn, results in production of defective protein). In one embodiment, the method is a method of producing a nonhuman animal that phenocopies symptoms of a genetic disease caused by altered splicing of a gene, referred to as the target (disease) gene, comprising administering to a nonhuman animal, such as a rodent (e.g., rat, mouse) splicing modulating synthetic antisense oligonucleotides (ASOs) that initiate or exacerbate mis-splicing of the target gene, thereby producing a nonhuman animal that comprises splicing modulating synthetic ASOs and phenocopies symptoms of the disease. The target gene can be a defective gene, such as a gene that predominantly expresses a defective protein (e.g., a truncated protein that results from alternative splicing of an exon, such as the truncated protein produced from the human SMN2 gene) or a normal (wild type) gene whose splicing can be altered by ASOs that target its pre-mRNA and cause (initiate) mis-splicing or increase (exacerbate) the extent to which it occurs. In one embodiment, the synthetic ASOs are complementary to a site on pre-mRNA of the target gene, the nonhuman animal is a nonhuman neonate and splicing modulating ASOs are administered by intracerebroventricular (ICV) injection, intrathecal administration or systematically, such as subcutaneously, intraperitoneally, intravenously or intramuscularly. The ASOs can be 2′-O-(2-methoxyethyl) (MOE) ASOs.

A further embodiment is a method of producing a nonhuman animal, such as a rodent, (e.g., a mouse, rat) that phenocopies a neurodegenerative disease, such as a human neurodegenerative disease, in which an essential endogenous gene (e.g., SMN1 gene) is lacking (is not present or functional in) and a human gene that is mis-spliced in humans who have or will develop a disease and rescues the nonhuman animal lacking the endogenous gene from lethality is present. In the method, synthetic ASOs that specifically alter the splicing pattern of the corresponding human gene (e.g., alter the splicing pattern of human SMN2) are administered, thereby producing a nonhuman animal in which the target gene (e.g., hSMN2) is mis-spliced more frequently than would otherwise occur and which phenocopies (exhibits characteristics of) the human disease (e.g., a neurodegenerative disease). In a specific embodiment, the animal is a rodent, such as any strain of mouse or rat.

In a specific embodiment, the method is a method of producing a nonhuman animal that phenocopies SMA, in which there is a splicing defect in SMN2 that results from alternative splicing that skips exon 7. One embodiment described herein is a method of producing a nonhuman animal (which can be a neonatal nonhuman animal, young (pre adult) nonhuman animal, adult nonhuman animal) that phenocopies intermediate “type II” spinal muscular atrophy (SMA). In one embodiment of the method, exon-skipping synthetic antisense oligonucleotides that inhibit transgenic human SMN2 splicing in the nonhuman animal are introduced into a mild “type III” SMA nonhuman animal in such a manner that they are delivered to the spinal cord, the locus of α-motor neurons. In one embodiment, the method comprises neonatal

intracerebroventricular (ICV) injection of exon-skipping synthetic antisense oligonucleotides that inhibit SMN2 splicing into a mild “type III” SMA nonhuman neonate, thereby producing a nonhuman animal that comprises synthetic exon-skipping ASOs (splicing modulating ASOs) and phenocopies intermediate type II SMA symptoms. The method can further comprise maintaining the resulting nonhuman neonate comprising the splicing modulating ASOs under conditions appropriate for it to live/grow. In one embodiment of the method, the nonhuman neonate is a mild “type III” SMA mouse (Smn^{-/-}; hSMN2^{+/0}) with four SMN2 copies, such as strain FVB.Cg-Tg(SMN2)2HungSmn1^{tm1Hung}/J. In any of the above embodiments of the method, ICV injection can be by means of a single injection or by means of multiple injections. The dose size and number of doses to be administered can be determined with reference to the desired outcome. In any of the embodiments, the intermediate type II SMA symptoms comprise at least one (one or more) of the following: SMA-like progressive motor dysfunction, SMA-like muscle weakness, SMA-like growth impairment, alpha-motor neuron degeneration and SMA-like shortened lifespan. The splicing modulating ASOs used in the method are complementary to a site or sites on SMN2 pre-mRNA, such as an exon 7 in SMN2 pre-mRNA, and inhibit (partially or completely) inclusion of exon 7 in SMN2 pre-mRNA. In a specific embodiment, the splicing modulating ASOs modified ASOs, such as 2′-O-(2-methoxyethyl) (MOE) ASOs or other modified ASOs (e.g., 2′-MOE oligonucleotides with phosphorothioate backbone, some or all 5-methylcytosines). They can be, for example, modified ASO-20-37, modified ASO-23-42, a combination of modified ASO-20-37 and modified ASO-23-42, modified oligonucleotides sufficiently similar in sequence to the nucleotide sequence of ASO-20-37 or ASO-23-4 that they exhibit substantially the same ability to hybridize to SMN2 pre-mRNA as evidenced by modified ASO-20-37 or modified ASO-23-42. Typically the ASO will have 100% sequence complementarity to the target region of the pre-mRNA. Alternatively, one or a few wobble base pairs (G-U) or mismatches can be included and maintain specific binding to target RNA.

A further embodiment is a method of producing a rodent that phenocopies intermediate “type II” spinal muscular atrophy (SMA) symptoms. The method comprises injecting synthetic exon-skipping ASOs by intracerebroventricular (ICV) injection into a mild “type III” SMA rodent neonate, in at least one dose sufficient to inhibit splicing of exon 7 of SMN2 pre-mRNA and result in skipping of SMN2 exon 7, thereby producing a rodent neonate that comprises synthetic exon-skipping ASOs (splicing modulating ASOs) and phenocopies intermediate type II SMA symptoms. The rodent can be a neonate, such as a neonatal mouse. If that is the case, the result is a rodent (e.g., mouse) neonate comprising the synthetic exon-skipping ASOs. The resulting rodent neonate is maintained under conditions sufficient for it to survive and grow, thus producing an older (young/preadult or adult) rodent model that phenocopies intermediate “type II” SMA symptoms.

Also described herein are synthetic antisense oligonucleotides (ASOs) that are complementary to [and hybridize to] a site on SMN2 pre-mRNA and inhibit splicing of exon 7 of SMN2 gene. Such synthetic ASOs can be injected intracerebroventricularly in a mild “type III” SMA nonhuman neonate in order to produce an intermediate “type II” SMA nonhuman neonate. In one embodiment, such synthetic splicing modulating ASOs are complementary to a site on SMN2 pre-mRNA that comprises: AAGAAGGAAGGTGCTCAC (SEQ ID NO.: 13). In a further embodiment, they are complementary to a sufficient region of AAGAAGGAAGGTGCTCAC (SEQ ID NO.: 13) that they hybridize with substantially the same specificity as that of ASOs that are complementary to the entire sequence. The synthetic ASOs can be modified and, for example, can comprise 2′-O-(2-methoxyethyl) modification of ribose and a phosphorothioate backbone. The synthetic splicing modulating ASOs can comprise (a) 5′-GTGAGCACCTTCCTTCTT-3′ (SEQ ID NO.: 10); (b) 5′-GGAATGTGAGCACCTTCCTT-3′ (SEQ ID NO.: 11); (c) a sequence sufficiently similar to 5′-GTGAGCACCTTCCTTCTT-3′ (SEQ ID NO.: 10) that the synthetic splicing modulating ASO hybridizes to the site on SMN2 pre-mRNA to which 5′-GTGAGCACCTTCCTTCTT-3′ hybridizes and inhibits splicing of exon 7 of SMN2 gene when injected intracerebroventricularly in a mild “type III”SMA nonhuman neonate; (d) a sequence sufficiently similar to 5′-GTGAGCACCTTCCTTCTT-3′ (SEQ ID NO.: 11) that the synthetic ASO hybridizes to the site on SMN2 pre-mRNA to which 5′-GTGAGCACCTTCCTTCTT-3′ hybridizes and inhibits splicing of exon 7 of SMN2 gene when injected intracerebroventricularly in a mild “type III” SMA nonhuman neonate; or (e) a combination of any 2, 3 or 4 of (a)-(d).

Also the subject of this work is a synthetic splicing modulating antisense oligonucleotide (ASO) that specifically binds to a SMN2 pre-mRNA cis-acting element by base pairing with the cis-acting element, inhibits splicing of exon 7 of SMN2 gene and causes reduced SMN protein expression in a mild “type III” SMA mouse when injected intracerebroventricularly in a mild “type III” SMA mouse neonate. In one embodiment, mild “type III” mouse into which splicing modulating ASOs are injected phenocopies intermediate type II SMA symptoms. A synthetic splicing modulating ASO can bind to a SMN1/2 splicing enhancing element, a 5′ splice site or a 3′ splice site.

The nonhuman animal models described herein are useful not only for studying the pathology and progression of the neurodegenerative disease they model, but also for identifying therapeutic agents useful for treating the disease, preventing the disease from occurring or reducing the extent to which the disease occurs or develops or progresses. The method of identifying a therapeutic agent comprises administering a candidate therapeutic agent (one to be assessed for its ability to treat the disease, prevent the disease from occurring or reducing the extent to which it develops or progresses) to a nonhuman model that phenocopies the disease of interest and assessing the effect of the candidate therapeutic agent on symptoms characteristic of the disease. If the symptoms are less after administration of the candidate therapeutic agent, the candidate therapeutic agent is a therapeutic agent for reducing SMA symptoms (treating the disease, preventing the disease from occurring or reducing the extent to which it develops or progresses).

In a specific embodiment, the method is a method of identifying therapeutic agents that reduce spinal muscular atrophy (SMA) symptoms, comprising administering a candidate therapeutic agent to a nonhuman model that phenocopies SMA symptoms and assessing the effect of the candidate therapeutic agent on the SMA symptoms, wherein if the symptoms are less after administration of the candidate therapeutic agent, the candidate therapeutic agent is a therapeutic agent for reducing SMA symptoms. In further specific embodiments, the nonhuman animal model of SMA is produced by any one of the methods described herein.

In short, Applicant has shown that a single ICV injection of ASO in neonatal mice can phenocopy a neurodegenerative disease and disease severity can be fine-tuned through dose-dependent effects on splicing. MOE ASOs induced SMA-like symptoms and histopathology that progressed irreversibly in the absence of therapy. TSUNAMI represents a useful alternative to genetic models based on stable or conditional knockout/rescue of disease-associated genes. When transgenic animal models are available, targeting the human transgene—as done here—offers the advantage that TSUNAMI can be used to test the same candidate therapeutics that would be used in the clinic. On the other hand, TSUNAMI should be applicable to any mouse strain or any animal species, provided that species-specific ASOs that appropriately modulate splicing of the target gene are used. Although described here is use of exon-skipping ASOs to elicit a splicing defect, and an exon-including ASO to correct it. The method can be readily adapted to promote other types of changes in splicing, as appropriate for each disease and target gene (Kole et al. 2012). The method described here should be compatible with other ASO chemistries, as long as they do not promote RNase H cleavage or RNAi. And, last but not least, TSUNAMI is not limited to modeling CNS diseases but can also be used to alter splicing of target genes expressed in various tissues, using the appropriate administration route, and if necessary, multiple dosing.

Mice have only one Smn gene, which is essential. A human SMN2 transgene rescues embryonic lethality, and Smn^{-/-} SMN2 mice have SMA-like phenotypes whose severity depends on the transgene copy number (Hsieh-Li, H. M. et al. Nat Genet 24, 66-70 (2000)), (Monani, U. R. et al. Hum Mol Genet 9, 333-339 (2000)). Severe ‘type I’ SMA mice harboring two SMN2 copies, with or without an additional SMNA7 cDNA transgene, die shortly after birth (−2 weeks)(Hsieh-Li, H. M. et al. Nat Genet 24, 66-70 (2000)), (Monani, U. R. et al. Hum Mol Genet 9, 333-339 (2000)), (Le, T. T. et al. Hum Mol Genet 14, 845-857 (2005)), (Riessland, M. et al. Hum Mol Genet 19, 1492-1506 (2010)), whereas mild ‘type III’ SMA mice harboring four SMN2 copies survive normally without paralysis, but develop tail and ear necrosis (Hsieh-Li, H. M. et al. Nat Genet 24, 66-70 (2000)). These strains, while extremely useful, fall short of providing phenotypically accurate models for intermediate ‘type II’ SMA that would allow detailed analyses of phenotype, pathophysiology, and therapeutic efficacy. Other SMA models with point mutations (smn213/-mice) or exon deletions (Smn^{F7163}, NSE-Cre mice) in murine Smn have intermediate phenotypes, including—1-month lifespan and progressive motor dysfunction with relevant pathological features; however, these genotypes are not present in SMA patients, and are incompatible with therapeutic strategies involving splicing correction or upregulation of SMN2.

Applicant previously showed that a 2′-0-(2-methoxyethyl) (MOE) therapeutic ASO (ASO-10-27) promotes exon 7 inclusion and rescues necrosis in type III mice; this ASO is metabolically stable in central nervous system (CNS) tissues, and corrects SMN2 splicing for more than 6 months (Hua, Y. et al. Genes Dev 24, 1634-1644 (2010)). Thus, ASOs can persistently control phenotypes through splicing modulation in the CNS. As described herein, Applicant has generated a practical mouse model of intermediate SMA, utilizing ASOs complementary to a different site on SMN2 pre-mRNA, which exacerbate exon 7 mis-splicing and promote pathogenesis.

Applicant demonstrates that intracerebroventricular (ICV) injection of splicing modulating (exon-skipping) ASOs in type III neonatal mice accurately phenocopies more severe SMA as a result of SMN2 mis-splicing. Treatment with therapeutic ASO-10-27 rescues splicing and ameliorates the SMA phenotypes. Furthermore, the SMN2 splicing defect is exacerbated as a result of starvation, which correlates with prognosis. ASOs can serve as useful tools to develop animal disease models by persistent modulation of splicing.

Described herein is exon-skipping ASO technology to phenocopy a motor-neuron disease by postnatally inhibiting SMN2 splicing in transgenic mice. Applicant successfully elicited phenotypes resembling core SMA symptoms, thus exacerbating the phenotype of a mild-SMA mouse model. To promote exon skipping, ASOs targeting splicing-enhancer sequences within exon 7 of SMN2 were selected, in part so as to avoid interference with the target site of ASO-10-27, in intron 7. This made it possible to perform splicing-rescue experiments using ASO-10-27.

Because SMA primarily affects α-motor neurons, Applicant assumed that SMN2 splicing in the brainstem and spinal cord would be critical. ICV administration was used for direct ASO delivery to the CNS, and a single injection had significant, dose-dependent effects on both SMN2 splicing and on the phenotypes. As with type I mice (Park et al. 2010a) or severe-SMA patients (Lunn and Wang 2008; Rudnik-Schoneborn et al. 2008), growth impairment and cardiac involvement likely influenced the prognosis of ASO-20-37-treated mice. With ˜1-month lifespan and overt SMA-like phenotypes that are partly shared by Smn^{2B/-} or Smn^{F7/Δ7}, NSE-Cre mice (Park et al. 2010a), these mice are especially useful for analyses of disease progression, physiological tests, and therapeutic efficacy.

Although ICV-administered MOE ASOs distribute throughout the CNS, they are partly cleared out of the CNS and accumulate in peripheral tissues. However, because of a combination of low dose, ASO dilution in plasma and tissues, much shorter half-life in peripheral tissues, and renal excretion (Crooke 2007), any pharmacological effects outside the CNS are likely minimal. Indeed, ASO-20-37 predominantly inhibited SMN2 splicing in the CNS, and resulted in α-motor neurons loss. The nuclear gem number—an indicator of SMN abundance (Lunn and Wang 2008)—decreased markedly in α-motor neurons, which could cause neuronal dysfunction. On the other hand, similarly to SMA Δ7 mice (Ling et al. 2012), ASO-20-37-treated mice exhibited NMJ defects. Until P16, the LC NMJs attained full innervation and the AChR clusters continued to mature normally. The reduced complexity of AChR topology at P30 might be attributed to rapid AChR turnover and/or impairment of further maturation due to NMJ denervation (Fambrough 1979) under conditions of α-motor neuron dysfunction after P16. Alternatively or additionally, ASO-20-37 might have a direct effect in muscle that alters endplate maturity. The ASO-mediated postnatal induction of SMA pathology reveals that SMN is required for α-motor-neurons after completion of their genesis around embryonic day 11 (Nornes and Carry 1978). Conversely, restoring SMN levels shortly after birth is therapeutically effective for severe SMA mice (Hua et al. 2011; Le et al. 2011; Lutz et al. 2011), consistent with a role of SMN in the postnatal development of motor neurons.

Compared with SC administration of therapeutic ASO-10-27, ICV administration considerably extended the lifespan and alleviated motor dysfunction of ASO-20-37-treated mice, implying that SMN2 splicing correction in the CNS is necessary and sufficient for phenotypic and histological amelioration. In contrast, Applicant recently found that neonatal SC administration of ASO-10-27 markedly extends the lifespan of type I mice (Hua et al. 2011). In that case, SMN restoration in peripheral tissues was necessary for efficient rescue, although an additional direct effect in the CNS was not excluded.

The apparent inconsistency between the two studies may reflect the fact that here SMN2 splicing was predominantly inhibited in the CNS, in contrast to the ubiquitous SMN2 splicing defect in type I mice (or human SMA). Another important difference is that here SMN depletion was induced postnatally, whereas it occurs embryonically in type I mice. Blood brain barrier (BBB) permeability in these mice might be more intact, compared with that in type I mice, which in the present study might reduce the CNS effects of systemically administered ASO-10-27. The present results suggest that the SMN level in the CNS is critical for the SMA phenotype, as well as its severity. On the other hand, SC administration of ASO-10-27 markedly rescued tail necrosis, whereas ICV administration only had a subtle effect, implying that peripheral SMN levels are more relevant for distal necrosis. Incomplete closure of the BBB in neonates (Stewart and Hayakawa 1987) and/or retrograde axonal transport of ASO (Crooke 2007) to spinal-cord neurons might also contribute to the rescue of tail necrosis by peripheral ASO administration, which would be consistent with the previous finding that ICV administration of therapeutic ASO-10-27 rescues tail necrosis in type III SMA mice (Hua et al. 2010).

SMN depletion in motor-neuron progenitors results in relatively mild SMA-like phenotypes, suggesting that other cells also contribute to SMA pathogenesis (Park et al. 2010b). The described ASOs do not exclusively target α-motor neurons (Hua et al. 2010; Passini et al. 2011) and, thus, their effects in other cells could also have contributing roles in this SMA model. Considering the potential developmental component of SMA, the ASO effects at different developmental stages also need to be addressed. Further analysis of the relationship between the spatial and/or temporal distribution of ASOs that promote correct or incorrect SMN2 splicing, and their pharmacological effects, should yield new insights into the roles of SMN in SMA pathogenesis, as well as its normal physiological functions.

SMN2 splicing deteriorated further in dying SMA mice and nutritional status impacted the splicing effects even in mild type III or wild-type transgenic mice in the absence of any ASO. Widespread splicing changes were reported in late-stage type I mice (Zhang et al. 2008; Baumer et al. 2009), and Applicant confirmed altered splicing of two representative genes, Uspl1 and Chod1, both in the ASO-induced SMA mice—especially at the terminal stage—and in food-deprived but untreated type III mice. The splicing changes uncovered in SMN2-transgene transcripts during late-stage SMA were not described in these studies, although in another study, SMN2 splicing was shown to decrease in type I mice at P1 (Jodelka et al. 2010; Ruggiu et al. 2012). Dying mice likely suffer from hypoxia; hypoxic stress induces reactive oxygen species generation, which inactivates the SMN complex (Wan et al. 2008). This complex plays fundamental roles in assembling snRNPs, which are required for splicing (Burghes and Beattie 2009), and low SMN levels result in decreased SMN2 exon 7 splicing through a feedback loop (Jodelka et al. 2010; Ruggiu et al. 2012). Thus, under critical dying conditions, including malnutrition and hypoxia, SMA may progress with gradual, widespread splicing alterations, in which the resulting SMN dysfunction and deficiency could be partly involved.

### EXAMPLES

### Methods and Materials

The Methods and Materials described throughout the application were used, as well as methods and materials known to those of skill in the art, in the work described herein.

Sequences of the uniform 2′-MOE ASOs with phosphorothioate backbone and 5-methyl cytosines used

| Antisense | | |
|---|---|---|
| oligonucleotide | Sequence | |
| Control ASO | 5′-TCATTTGCTTCATA | (SEQ ID NO. 9) |
| | CAGG-3′ | |
| ASO-20-37 | 5′-GTGAGCACCTTCCT | (SEQ ID NO. 10) |
| | TCTT-3′ | |
| ASO-23-42 | 5′-GGAATGTGAGCACC | (SEQ ID NO. 11) |
| | TTCCTT-3′ | |
| ASO-10-27 | 5′-TCACTTTCATAATG | (SEQ ID NO. 12) |
| | CTGG-3′ | |

### Oligonucleotide Synthesis

2′-MOE oligonucleotides with phosphorothioate backbone and all 5-methylcytosines were synthesized and purified as described (Hua, Y. et al. Genes Dev 24, 1634-1644 (2010)). ASOs were dissolved in saline and adjusted to the desired concentration. The ASO sequences are shown above.

### Animals

Mouse protocols were approved by Cold Spring Harbor Laboratory's Institutional Animal Care and Use Committee. Type III SMA mice (Smn^{-/-}; hSMN2^{+/+}) with four SMN2 copies were purchased from The Jackson Laboratory (strain FVB.Cg-Tg(SMN2)2Hung smn1^{tm1Hung}/J). Type I SMA mice (Smn^{-/-}; SMN2^{+/0}) harboring two SMN2 copies were generated as described (Gogliotti et al Biochem Biophys Res Commun 391m 517-522 (2010); Riessland, M. et al. Hum Mol Genet 19, 1492-1506 (2010)). After weaning, mice were fed a normal chow diet (PicoLab Rodent Diet 20, LabDiet) and a nutrient-fortified water gel diet (DietGel Recovery, ClearH2O).

### Neonatal Injection

For ICV injection, P1 or P2 mice were cryo-anesthetized on ice, and 2 μl of ASO in saline was injected into the right lateral ventricle using a 5-μl micro-syringe (Hamilton) and a 33-gauge needle. The coordinates for injection were: 1 mm lateral from the sagittal suture, 2 mm anterior from the lambdoid suture, and 2 mm deep. ICV injections were well tolerated. Fast Green FCF (0.01% (w/v); Sigma-Aldrich) was included in the ASO solution, so that the shape of both lateral and fourth ventricles could be visualized when the ICV injection was successful. No leakage was observed around the skull surface. For subcutaneous injection, 1.5-8.0 μl of 50 μg/g of ASO in saline—with the volume adjusted according to the body weight—was injected into the dorsal skin using a 10-μl micro-syringe (Hamilton) and a 33-gauge needle.

### Radioactive RT-PCR and Western Blotting

For each group, RT-PCR with ³²P-dCTP was performed with tissues from three mice. Total RNA extraction and RT-PCR were performed to analyze SMN2 transcripts as described (Hua, Y., et al. PLoS Biol 5, e73 (2007)), (Hua, Y., et al. Am J Hum Genet 82, 834-848 (2008)). Both radioactive RT-PCR and real-time PCR had previously been used to quantitate SMN2 exon-7 included and skipped isoforms, with entirely consistent results (Hua et al. 2010). To analyze Uspl1 or Chod1 splicing patterns, or Aif1 expression level, the following PCR primers were used:

Uspl1: forward, 5′-AGTTCGGGTCCACTGTATGC-3′ (SEQ. ID No. 1) and reverse, 5′-GGCTTACTTGGAGTGGGACA-3′(SEQ. ID No. 2),
Chod1: forward, 5′-AAGCCGTATCTTACAAACCAACCT-3′ (SEQ. ID No. 3) and reverse, 5′-CCACTTTCCTTCCTCGTGCT-3′(SEQ. ID No. 4),
Aif1: forward, 5′-CAGCAATGATGAGGATCTGC-3′(SEQ. ID No. 5) and reverse, 5′-GTTTCTCCAGCATTCGCTTC-3′(SEQ. ID No. 6).
Gapdh mRNA levels were analyzed using the following primers: forward, 5′-CGTCCCGTAGACAAAATGGT-3′(SEQ. ID No. 7) and reverse, 5′-GAATTTGCCGTGAGTGGAGT-3′(SEQ. ID No. 8). PCR products were analyzed by native PAGE and phosphorimage detection (FLA-5100, Fujifilm). Band intensities were quantitated with Multi Gauge software (Fujifilm). The signal intensity of each band was normalized according to its GC content.

Total protein was extracted from spinal cord and analyzed by western blotting. The blots were probed with monoclonal antibody (mAb) specific for human SMN (SMN-KH), or anti-α-tubulin mAb (Sigma-Aldrich), followed by IRDye-conjugated secondary antibody (LI-COR Biosciences). Protein bands were detected and quantitated with an Odyssey Infrared Imaging System (LI-COR Biosciences) as described (Hua et al. 2010; Hua et al. 2011), (Hua, Y. et al. Genes Dev 24, 1634-1644 (2010); Hua, Y. et al. Nature 478, 123-126 (2011)).

### Cell Culture and Transfection

Type I SMA patient fibroblasts (GM03813, Coriell Institute) were cultured as described (Hua, Y., et al. Am J Hum Genet 82, 834-848 (2008)). ASOs were transfected using Lipofectamine 2000 (Invitrogen).

### Neurological and Behavioral Tests

The righting reflex was assessed at P8, P10, and P12. Pups were placed on their side, and the time elapsed until they placed all four paws stably on the ground was recorded.

A rotarod task was carried out using a RotaRodIV instrument (AccuScan). The time to fall from a 7-rpm-rotating rod was measured until an arbitrary limit of 180 sec. Ten sequential trials were performed, and the longest time was recorded.

Grip strength was measured using a Grip Strength Meter (Columbus Instruments). Mouse forelimbs were placed on a triangle bar, and once they grasped it, the mice were pulled back horizontally until they released it. Six sequential trials were performed, and the highest value was recorded.

Phenotypic characterization of mouse behavior was performed using a video-based behavior-recognition platform (Steele, A. D., et al. Proc Natl Acad Sci USA 104, 1983-1988 (2007)). Mice were recorded throughout a 12-hour dark phase, and the data were analyzed using HomeCageScan software (Clever Sys.).

### Immunostaining and Histology

Formalin-fixed, paraffin-embedded spinal cord was cut into 6-micron sections. After antigen retrieval with citrate buffer (pH 6.0), each section was incubated with goat anti-choline acetyltransferase antibody (ChAT) (1:500; Millipore) Immunoreactivity was detected by ImmPress anti-goat IgG antibody (Vector Laboratories) and DAB substrate-chromogen (Dako, Denmark), followed by hematoxylin counterstaining ChAT-positive α-motor neurons in the anterior horn with a single nucleolus within the nucleus (>350 μm²) (Friese et al. 2009) were counted in 6-micron, 24 serial sections of L1-L2 spinal cord. To ensure consistent and non-redundant counting, the total neuron counts were determined in every 8th section for 3 sections, and the counts of the 8 groups were averaged. All the steps from harvesting through staining of spinal cords were processed in parallel, so as to reduce artifactual variation between experimental samples. ASO cellular uptake was assessed with antibody against the phosphorothioate backbone. For dual staining for SMN and ChAT, the section was incubated with anti-ChAT antibody (1:500), followed by Alexa Fluor 594 IgG antibody (Invitrogen), and anti-SMN antibody (1:500; BD Biosciences), followed by Alexa Fluor 488 IgG antibody (Invitrogen). Nuclear gems located within 100 α-motor neurons were counted in L1-L2 spinal cord.

For NMJ staining, after perfusing and postfixing with 4% paraformaldehyde, whole muscles were dissected and teased into layers, 5-10 fibers thick. NMJs were immunolabeled with anti-neurofilament (1:2000; Chemicon) for nerves, anti-synaptophysin (1:200; Invitrogen) for presynaptic terminals, and Alexa Fluor 594-conjugated α-bungarotoxin (Invitrogen) for acetylcholine receptors. The proportion of fully innervated NMJs was quantified. Fully innervated NMJs were defined by the complete overlap of presynaptic (i.e., synaptophysin) and postsynaptic (α-bungarotoxin) labeling. Endplate maturity was assessed by AChR topology, as described (Kummer et al. J. Cell Biol. 164, 1077-1087 (2004)), and the percentage of endplates with mature AChR topology was quantified. For each sample, at least 100 NMJs were evaluated from random visual fields of the whole mount. Flash-frozen quadriceps muscle and formalin-fixed heart were cut into 14- and 7-micron sections, respectively, at the level of each maximum transection, for H&E staining. Maximum heart interventricular septal thickness was measured in these sections. Images were acquired with an Axio Observer.Z1 microscope and an LSM 710 confocal microscope (Carl Zeiss, Germany) for bright-field and immunofluorescence imaging, respectively.

### Statistical Analysis

Data was analyzed using two-tailed t-tests, and P values≦0.05 were considered to be statically significant. For analysis of home-cage behaviors, the Friedman test, a non-parametric version of ANOVA (Hollander, M. & Wolfe, D. A. (John Wiley & Sons, Inc., Hoboken, N.J.; 1973)) implemented in MATLAB was used. Kaplan-Meier survival curves were prepared with Prism 5 (GraphPad Software) and statistical significance was calculated with the logrank (Mantel-Cox) test (Bland, J. M. & Altman, D. G. BMJ 328, 1073 (2004)). The various histograms and plots show mean values±standard deviation.

### Results

### ASO-Inhibition of SMN2 Splicing

Based on a previous screen of ASOs tiled along SMN2 exon 7 (Hua et al. 2007), Applicant designed two new ASOs, ASO-20-37 and ASO-23-42 (FIG. 1A; Supplemental Table S1). Their target sites in exon 7 comprise potent enhancer elements (Lunn and Wang 2008). MOE phosphorothioate ASOs, which do not trigger RNase H cleavage or RNAi (Crooke 2007), were used. Their splicing-inhibitory potencies were initially assessed by RT-PCR in type I SMA patient fibroblasts; both ASOs considerably decreased exon 7 inclusion (˜10-fold and ˜30-fold reduction by 100 nM ASO-20-37 and ASO-23-42, respectively).

These ASOs are specific for SMN1/2, and, in order to determine if it is possible to phenocopy SMA pathology, they were used to target an SMN2 transgene in type III SMA mice (Smn^{-/-} SMN2^{+/+}) with normal motor function and lifespan (Hsieh-Li et al. 2000). These phenotypes were followed to determine if they could be exacerbated by ASO-mediated splicing modulation. Severe-SMA patients show initial symptoms during the newborn and early-infant periods (Lunn and Wang 2008). In addition, the blood-brain bather (BBB) limits the access of systemically administered MOE ASO to the CNS (Crooke 2007; Hua et al. 2008; Hua et al. 2011). Therefore, they were introducted by direct administration into the cerebroventricular space in neonatal mice, which delivers ASOs via cerebrospinal fluid (CSF) to the brainstem and spinal cord, the loci of α-motor neurons (Hua et al. 2010). A single 20-μg (14.2 mg/kg) injection at postnatal day 1 (P1) was used and SMN2 splicing at P7 in the spinal cord was analyzed. Applicant focused analysis on the thoracic-cord region, but also observed similar effects throughout the spinal cord. Both ASO 20-37 and ASO-23-42 potently decreased exon 7 inclusion (˜4-fold and ˜3-fold reduction, respectively), whereas a mismatch control ASO had no effect. ASO-20-37 was slightly more effective than ASO-23-42, whereas the opposite was observed in patient fibroblasts, reflecting size-dependent differences in ASO uptake between cell transfection and spontaneous in vivo delivery, species-dependent pharmacokinetics, or different genetic backgrounds, cell types, and/or developmental stages, each of which can affect alternative-splicing regulation (Cartegni et al. 2002) Immunoblotting revealed a ˜5-fold reduction in SMN expression at P7 by ASO-20-37 treatment.

Focusing on ASO-20-37, Applicant found that it inhibited exon 7 inclusion in a dose-dependent manner. Because of CSF clearance, ICV-injected ASO partially distributes to peripheral tissues (Hua et al. 2010; Hua et al. 2011). Therefore, its effects were analyzed in other tissues. For muscle, Applicant analyzed proximal quadriceps, because motor tests pointed to a decline in hind-limb strength that limits essential behaviors. Exon 7 splicing inhibition was comparable in the brain and spinal cord, whereas in peripheral tissues ASO-20-37 had little or no effect.

MOE chemistry minimizes neuroinflammation and is well tolerated (Crooke 2007; Bennett and Swayze 2010; Hua et al. 2010), consistent with the very subtle increase in Aif1 mRNA expression—a macrophage-activation marker—in P14 spinal cord of mice injected with 20 μg ASO-20-37. Aif1 expression increased at P35, probably reflecting the disease process.

### SMN2 Splicing Deterioration in Terminal-Stage SMA

To determine the duration of the ASO effect, Applicant measured SMN2 splicing at P30. Compared with the effects at P7, exon 7 inclusion was further suppressed in the spinal cord (˜15-fold and ˜5-fold reduction by treatment with 20 μg ASO-20-37 and ASO-23-42, respectively). Accordingly, there was a further reduction in SMN protein (˜12-fold). In contrast to P7 mice, Applicant observed increased mis-splicing in peripheral tissues at P30, despite minimal ASO uptake. Simultaneous skipping of exons 7 and 5 also increased in 20-μg or 30-μg ASO-20-37-treated mice, suggesting indirect inhibition of SMN2 splicing in response to ASO treatment. Furthermore, extensive exon 7 skipping occurred systemically at P35, the terminal stage for mice injected with 20 μg ASO. The finding that the level of SMN2 splicing at P14 was similar to that at P7 suggests that SMN2 splicing is subsequently suppressed at P30-35 because of the end-stage disease state, rather than by a persistent ASO effect. Consistent with this interpretation, neonatal ICV injection of therapeutic ASO-10-27 with the same chemical modifications had reduced or no effect in P30 spinal cord, liver, or heart, compared with that at P7 (Hua et al. 2010).

As a further test, SMN2 splicing in untreated P1 or P7-8 type I SMA mice (Smn^{-/-} SMN2^{+/0}) was compared with that in type III mice (Smn^{-/-} SMN2^{+/+}) or healthy mice (Smn^{+/+} SMN2^{+/0} and Smn^{+/-} SMN2^{+/0}). At P7-8, type I mice typically become cachectic and moribund, and exon 7 inclusion in their spinal cord decreased at this time, whereas it increased in type III or healthy mice. A similar trend was observed in heart between type I and III mice.

These results suggest that SMN2 splicing is suppressed under cachexia, perhaps due to nutritional deficiency, considering that dying SMA mice have poor food intake and extensive weight loss. To directly test this hypothesis, untreated type III mouse pups or adults were subjected to food deprivation; after 48 h, SMN2 splicing was markedly inhibited in both the CNS and peripheral tissues, especially in pups. Remarkably, when these pups were fed again, SMN2 splicing was restored, demonstrating a nutritional effect on splicing. Importantly, this inhibition of SMN2 splicing was also observed in the spinal cord of food-deprived wild-type transgenic mice (Smn^{+/+} SMN2^{+/+}).

Widespread splicing abnormalities were reported in late-stage type I mice, although whether these are the cause or consequence of SMA progression is controversial (Zhang et al. 2008; Baumer et al. 2009). Applicant analyzed previously reported splicing alterations of Uspl1 and Chod1 (Zhang et al. 2008; Baumer et al. 2009) in the spinal cord of P7 or P30 ASO-20-37-treated mice. RT-PCR demonstrated abnormal splicing of both pre-mRNAs, especially at P30. In contrast, splicing of these genes was not altered at P7 in normal mice (Smn^{+/+}) treated with ASO-20-37. Splicing was also altered in food-deprived but otherwise untreated type III mice, indicating that these splicing abnormalities are not a direct consequence of ASO-20-37 treatment.

### ASO Induction of SMA-Like Phenotypes

Both ASOs elicited ‘SMA-like’ phenotypes. ICV injection of 20 μg ASO at P1 strikingly shortened the lifespan of type III SMA mice, and ASO-20-37 was more potent than ASO-23-42 (median survival 33 versus 41 days; P<0.0001). Focusing on ASO-20-37, Applicant observed a dose-dependent reduction in survival and weight gain. There was no change in the onset or progression of tail necrosis. With respect to motor dysfunction—a cardinal SMA symptom—the mice exhibited a staggering gait with short strides that became apparent around weaning. Dose-dependent motor impairment was evident in Rotarod and grip-strength tests. Moreover, video-based analysis of home-cage behaviors showed declined locomotor activity, including decreases in standing, eating, and hanging. These physiological tests were mainly performed at P25-26, when mice injected with 20 μg ASO-20-37 grew large enough to perform the rotarod task. Although ASO-20-37-treated mice had a normal righting reflex until P12 and maintained grip strength at P15 and P22, once the abnormal gait occurred, their locomotor activity declined progressively, culminating in death. Both ASO-20-37- and ASO-23-42-treated mice also displayed systemic tremor.

### Supportive SMA Histological Features

α-motor neuron degeneration is the pathological hallmark of SMA (Lunn and Wang 2008). In ASO-20-37-treated mice, immunohistochemistry (IHC) of lumbar spinal cord showed significant loss of choline acetyltransferase (ChAT)-positive α-motor neurons at P30 but not at P16. In contrast, there was a small but not significant loss in cervical spinal cord at P30. IHC with antibody against the phosphorothioate backbone showed that motor neurons in lumbar spinal cord still have internalized ASO at P30. SMN-positive nuclear gem counts—which correlate directly with functional SMN protein level, and inversely with SMA severity (Lunn and Wang 2008)—decreased markedly. This SMN decrease probably triggers neuronal dysfunction and subsequent degeneration.

Groups of atrophic fibers are seen in quadriceps of type I mice (Hsieh-Li et al. 2000). However, Applicant did not observe this feature, which would have been indicative of a pre-pathological stage preceding rapid disease progression (Dubowitz and Sewry 2007). Recent reports described structural and electrophysiological defects in the neuromuscular junction (NMJ) in severe SMA (Burghes and Beattie 2009). Applicant analyzed NMJ pathology in several vulnerable muscles that are affected in SMA Δ7 mice (Ling et al. 2012). Acetylcholine receptor (AChR) clusters at the NMJ are initially plaque-shaped; later, perforated plaque, fold/C-shaped, branched, and pretzel-shaped structures become successively more prevalent as development proceeds (Kummer et al. 2004). In ASO-20-37-treated mice at P30, immunofluorescence staining for NMJs in longissimus capitus (LC) and serratus posterior inferior (SPI) showed smaller AChR clusters with reduced topological complexity; there were fewer mature-pretzel and branched forms, and more of the less-mature forms. Moreover, fully innervated NMJs (see Materials and methods) were markedly reduced in LC. There was a small reduction in the extent of innervation in splenius capitus, but little or no reduction in SPI and extensor digitorum longus (Supplemental Fig. S7C). The preservation of α-motor neuron counts, AChR topology, and NMJ innervation in LC at P16 is consistent with the progressive nature of the disease. Taken together, these results show that ASO-20-37-treated mice recapitulated the characteristic SMA pathology.

Finally, there is cardiac involvement in severe SMA (Rudnik-Schoneborn et al. 2008; Bevan et al. 2010), but Applicant detected no evidence of inflammation in heart sections at P30, such as cell infiltration and increased interstitial fibrosis. At the terminal stage, however, some ASO-20-37-treated mice manifested paw and face edema, and tachypnea, indicative of cardiac and/or respiratory failure, despite only faint ASO uptake in heart, lung, and diaphragm at P30. To characterize the cardiac phenotype, hearts were harvested and weighed at P30. Consistent with a study of type I mice (Bevan et al. 2010), the heart mass was reduced, even when normalized to femoral length. In addition, there was an increase in interventricular septal thickness, normalized to the heart mass, suggesting cardiac involvement.

### Amelioration of SMA-Like Symptoms by a Therapeutic ASO

Applicant previously reported an ASO complementary to positions +10 to +27 in SMN2 intron 7 (ASO-10-27, SEQ ID NO.: 12) that blocks a splicing silencer element, strongly increasing exon 7 inclusion (Hua et al. 2008). This therapeutic ASO-10-27, when administered by neonatal ICV and/or systemic subcutaneous (SC) injection, effectively rescues both type I and type III mouse phenotypes (Hua et al. 2010; Hua et al. 2011; Passini et al. 2011). Here, Applicant tested whether it could also rescue SMN2 splicing in ASO-20-37-treated mice, and thus influence the phenotype. After ICV injection of 20 μg ASO-20-37 at P1, ASO-10-27 was administered by ICV or systemic SC injection. ICV injection of 20 μg ASO-10-27 at P2 markedly extended the lifespan (median survival >100 days, P<0.0001) with improved weight gain and slightly delayed onset of tail necrosis. The treatment also improved motor function, and correspondingly prevented α-motor neuron loss and preserved normal AChR topology and NMJ innervation. In contrast, SC injection of ASO-10-27 (50 or 200 μg/g/day at both P2 and P3) only had a slight effect on survival (median survival, 50 μg/g/day: 40 days, P<0.0001; 200 μg/g/day: 41 days, P<0.0001). However, it moderately improved weight gain and motor function, and markedly rescued tail necrosis. RT-PCR showed that ICV injected ASO-10-27 restored SMN2 splicing in the spinal cord to the control level, but had little or no effect in liver, heart, and muscle, whereas SC injection only had a small effect in the spinal cord, but significant effects in peripheral tissues, especially in liver, as previously reported (Hua et al. 2008; Hua et al. 2010; Hua et al. 2011). These results indicate that the extent of exon 7 skipping in the spinal cord is the main determinant of the SMA-like symptoms induced by ICV administration of ASO-20-37, and they also support ASO-10-27 as a drug candidate.

### REFERENCES

Aartsma-Rus A. 2010. Antisense-mediated modulation of splicing: therapeutic implications for Duchenne muscular dystrophy. RNA Biol 7: 453-461.
Baumer D, Lee S, Nicholson G, Davies J L, Parkinson N J, Murray L M, Gillingwater T H, Ansorge O, Davies K E, Talbot K. 2009. Alternative splicing events are a late feature of pathology in a mouse model of spinal muscular atrophy. PLoS Genet 5: e1000773.
Bennett C F, Swayze E E. 2010. RNA targeting therapeutics: molecular mechanisms of antisense oligonucleotides as a therapeutic platform. Annu Rev Pharmacol Toxicol 50: 259-293.
Bevan A K, Hutchinson K R, Foust K D, Braun L, McGovern V L, Schmelzer L, Ward J G, Petruska J C, Lucchesi P A, Burghes A H et al. 2010. Early heart failure in the SMNDelta7 model of spinal muscular atrophy and correction by postnatal scAAV9-SMN delivery. Hum Mol Genet 19: 3895-3905.
Burghes A H, Beattie C E. 2009. Spinal muscular atrophy: why do low levels of survival motor neuron protein make motor neurons sick? Nat Rev Neurosci 10: 597-609.
Cartegni L, Chew S L, Krainer A R. 2002. Listening to silence and understanding nonsense: exonic mutations that affect splicing. Nat Rev Genet 3: 285-298.
Crooke S T. 2007. Antisense Drug Technology: Principles, Strategies, and Applications CRC Press, Boca Raton, Fla.
Dubowitz V, Sewry C A. 2007. Muscle Biopsy: A Practical Approach. Elsevier, Amsterdam, Netherlands.
Fambrough D M. 1979. Control of acetylcholine receptors in skeletal muscle. Physiol Rev 59: 165-227.
Fletcher S, Adams A M, Johnsen R D, Greer K, Moulton H M, Wilton S D. 2010. Dystrophin isoform induction in vivo by antisense-mediated alternative splicing. Mol Ther 18: 1218-1223.
Friese A, Kaltschmidt J A, Ladle D R, Sigrist M, Jessell T M, Arber S. 2009. Gamma and alpha motor neurons distinguished by expression of transcription factor Err3. Proc Natl Acad Sci USA 106: 13588-13593.
Gogliotti R G, Hammond S M, Lutz C, Didonato C J. 2010. Molecular and phenotypic reassessment of an infrequently used mouse model for spinal muscular atrophy. Biochem Biophys Res Commun 391: 517-522.
Hsieh-Li H M, Chang J G, Jong Y J, Wu M H, Wang N M, Tsai C H, Li H. 2000. A mouse model for spinal muscular atrophy. Nat Genet 24: 66-70.
Hua Y, Sahashi K, Hung G, Rigo F, Passini M A, Bennett C F, Krainer A R. 2010. Antisense correction of SMN2 splicing in the CNS rescues necrosis in a type III SMA mouse model. Genes Dev 24: 1634-1644.
Hua Y, Sahashi K, Rigo F, Hung G, Horev G, Bennett C F, Krainer A R. 2011. Peripheral SMN restoration is essential for long-term rescue of a severe spinal muscular atrophy mouse model. Nature 478: 123-126.
Hua Y, Vickers T A, Baker B F, Bennett C F, Krainer A R. 2007. Enhancement of SMN2 exon 7 inclusion by antisense oligonucleotides targeting the exon. PLoS Biol 5: e73.
Hua Y, Vickers T A, Okunola H L, Bennett C F, Krainer A R. 2008. Antisense masking of an hnRNP A1/A2 intronic splicing silencer corrects SMN2 splicing in transgenic mice. Am J Hum Genet 82: 834-848.
Jodelka F M, Ebert A D, Duelli D M, Hastings M L. 2010. A feedback loop regulates splicing of the spinal muscular atrophy-modifying gene, SMN2. Hum Mol Genet 19: 4906-4917.
Kole R, Krainer A R, Altman S. 2012. RNA therapeutics: beyond RNA interference and antisense oligonucleotides. Nat Rev Drug Discov 11: 125-140.
Kummer T T, Misgeld T, Lichtman J W, Sanes J R. 2004. Nerve-independent formation of a topologically complex postsynaptic apparatus. J Cell Biol 164: 1077-1087.
Le T T, McGovern V L, Alwine I E, Wang X, Massoni-Laporte A, Rich M M, Burghes A H. 2011. Temporal requirement for high SMN expression in SMA mice. Hum Mol Genet 20: 3578-3591.
Ling K K, Gibbs R M, Feng Z, Ko C P. 2012. Severe neuromuscular denervation of clinically relevant muscles in a mouse model of spinal muscular atrophy. Human molecular genetics 21: 185-195.
Lunn M R, Wang C H. 2008. Spinal muscular atrophy. Lancet 371: 2120-2133.
Lutz C M, Kariya S, Patruni S, Osborne M A, Liu D, Henderson C E, Li D K, Pellizzoni L, Rojas J, Valenzuela D M et al. 2011. Postsymptomatic restoration of SMN rescues the disease phenotype in a mouse model of severe spinal muscular atrophy. J Clin Invest 121: 3029-3041.
Muntoni F, Wood M J. 2011. Targeting RNA to treat neuromuscular disease. Nat Rev Drug Discov 10: 621-637.
Nornes H O, Carry M. 1978. Neurogenesis in spinal cord of mouse: an autoradiographic analysis. Brain Res 159: 1-6.
Park G H, Kariya S, Monani U R. 2010a. Spinal muscular atrophy: new and emerging insights from model mice. Curr Neurol Neurosci Rep 10: 108-117.
Park G H, Maeno-Hikichi Y, Awano T, Landmesser L T, Monani U R. 2010b. Reduced survival of motor neuron (SMN) protein in motor neuronal progenitors functions cell autonomously to cause spinal muscular atrophy in model mice expressing the human centromeric (SMN2) gene. J Neurosci 30: 12005-12019.
Passini M A, Bu J, Richards A M, Kinnecom C, Sardi S P, Stanek L M, Hua Y, Rigo F, Matson J, Hung G et al. 2011. Antisense oligonucleotides delivered to the mouse CNS ameliorate symptoms of severe spinal muscular atrophy. Sci Transl Med 3: 72ra18.
Riessland M, Ackermann B, Forster A, Jakubik M, Hauke J, Garbes L, Fritzsche I, Mende Y, Blumcke I, Hahnen E et al. 2010. SAHA ameliorates the SMA phenotype in two mouse models for spinal muscular atrophy. Hum Mol Genet 19: 1492-1506.
Rudnik-Schoneborn S, Heller R, Berg C, Betzler C, Grimm T, Eggermann T, Eggermann K, Wirth R, Wirth B, Zerres K. 2008. Congenital heart disease is a feature of severe infantile spinal muscular atrophy. J Med Genet 45: 635-638.
Ruggiu M, McGovern V L, Lotti F, Saieva L, Li D K, Kariya S, Monani U R, Burghes A H, Pellizzoni L. 2012. A role for SMN exon 7 splicing in the selective vulnerability of motor neurons in spinal muscular atrophy. Molecular and cellular biology 32: 126-138.
Sandy P, Ventura A, Jacks T. 2005. Mammalian RNAi: a practical guide. Biotechniques 39: 215-224.
Steele A D, Jackson W S, King O D, Lindquist S. 2007. The power of automated high-resolution behavior analysis revealed by its application to mouse models of Huntington's and prion diseases. Proc Natl Acad Sci USA 104: 1983-1988.
Stewart P A, Hayakawa E M. 1987. Interendothelial junctional changes underlie the developmental ‘tightening’ of the blood-brain barrier. Brain Res 429: 271-281.
Wan L, Ottinger E, Cho S, Dreyfuss G. 2008. Inactivation of the SMN complex by oxidative stress. Mol Cell 31: 244-254.
Zhang Z, Lotti F, Dittmar K, Younis I, Wan L, Kasim M, Dreyfuss G. 2008. SMN deficiency causes tissue-specific perturbations in the repertoire of snRNAs and widespread defects in splicing. Cell 133: 585-600.

1. A method of producing a non-human animal that phenocopies a human disease caused by altered splicing of a human disease gene, comprising:
administering splicing modulating antisense oligonucleotides (ASOs) that cause mis-splicing of the human disease gene to a non-human neonate comprising (a) the human disease gene or (b) an endogenous gene corresponding to the human disease gene,
wherein the splicing modulating ASOs are administered in sufficient quantity and via a route effective to produce a non-human animal that phenocopies the human disease.

2-48. (canceled)

49. The method of claim 1, wherein the non-human neonate is a rodent neonate.

50. The method of claim 49, wherein the rodent neonate is a mouse neonate.

51. The method of claim 1, wherein the age of the non-human neonate is postnatal day 1 or postnatal day 2.

52. The method of claim 1, wherein the human disease is a genetic disease, a neurodegenerative disease, cancer, a psychiatric disorder, a metabolic disorder, a cardiovascular disorder or a premature aging disorder.

53. The method of claim 52, wherein the human disease is a genetic disease.

54. The method of claim 53, wherein the genetic disease is spinal muscular atrophy.

55. The method of claim 1, wherein the splicing modulating ASOs are complementary to a region within pre-mRNA encoded by the human disease gene that includes a mutation that alters pre-mRNA splicing and causes the human disease.

56. The method of claim 1, wherein the splicing modulating ASOs are 2′-O-(2-methoxyethyl) splicing modulating ASOs.

57. The method of claim 1, wherein the splicing modulating ASOs are administered by intracerebroventricular injection or by subcutaneous injection.

58. A method of producing a mouse that phenocopies a human genetic disease caused by altered splicing of a human disease gene, comprising:
administering splicing modulating antisense oligonucleotides (ASOs) that cause mis-splicing of the human disease gene to a mouse neonate comprising (a) the human disease gene or (b) an endogenous gene corresponding to the human disease gene,
wherein the splicing modulating ASOs are administered in sufficient quantity and via a route effective to produce a mouse that phenocopies the human disease.

59. The method of claim 58, wherein the age of the mouse neonate is postnatal day 1 or postnatal day 2.

60. The method of claim 58, wherein the human genetic disease is spinal muscular atrophy.

61. The method of claim 58, wherein the splicing modulating ASOs are complementary to a region within pre-mRNA encoded by the human disease gene that includes a mutation that alters pre-mRNA splicing and causes the human disease.

62. The method of claim 58, wherein the splicing modulating ASOs are 2′-O-(2-methoxyethyl) splicing modulating ASOs.

63. The method of claim 58, wherein the splicing modulating ASOs are administered by intracerebroventricular injection or subcutaneous injection.

64. A method of producing a mouse that phenocopies human spinal muscular atrophy, comprising:
administering splicing modulating antisense oligonucleotides (ASOs) that cause mis-splicing of a human SMN2 gene to a mouse neonate comprising a human SMN2 gene,
wherein the splicing modulating ASOs are administered in sufficient quantity and via a route effective to produce a mouse that phenocopies human spinal muscular atrophy.

65. The method of claim 64, wherein the age of the mouse neonate is postnatal day 1 or postnatal day 2.

66. The method of claim 64, wherein the genotype of the mouse neonate is Smn2^{-/-} SMN2^{+/+}.

67. The method of claim 66, wherein the strain of the mouse neonate is FVB.Cg-Tg(SMN2)2HungSmn1^{tm1Hung}/J.

68. The method of claim 64, wherein the splicing modulating ASOs are 2′-O-(2-methoxyethyl) splicing modulating ASOs.

69. The method of claim 64, wherein the splicing modulating ASOs are complementary to a splicing-enhancer sequence within exon 7 of pre-mRNA encoded by the human SMN2 gene.

70. The method of claim 69, wherein the region comprises the nucleotide sequence AAGAAGGAAGGTGCTCAC (SEQ ID NO.: 13).

71. The method of claim 70, wherein the splicing modulating ASOs comprise splicing modulating ASOs that include the nucleotide sequence GTGAGCACCTTCCTTCTT (SEQ ID NO: 10), splicing modulating ASOs that include GGAATGTGAGCACCTTCCTT (SEQ ID NO: 11), or a combination of splicing modulating ASOs that include SEQ ID NO: 10 and splicing modulating ASOs that include SEQ ID NO: 11.


SEQUENCE LISTING (13 sequences)

SEQ ID NO: 1 - DNA, 20 bp, Artificial Sequence
  Synthetic oligonucleotide
agttcgggtccactgtatgc

SEQ ID NO: 2 - DNA, 20 bp, Artificial Sequence
  Synthetic oligonucleotide
ggcttacttggagtgggaca

SEQ ID NO: 3 - DNA, 24 bp, Artificial Sequence
  Synthetic oligonucleotide
aagccgtatcttacaaaccaacct

SEQ ID NO: 4 - DNA, 20 bp, Artificial Sequence
  Synthetic oligonucleotide
ccactttccttcctcgtgct

SEQ ID NO: 5 - DNA, 20 bp, Artificial Sequence
  Synthetic oligonucleotide
cagcaatgatgaggatctgc

SEQ ID NO: 6 - DNA, 20 bp, Artificial Sequence
  Synthetic oligonucleotide
gtttctccagcattcgcttc

SEQ ID NO: 7 - DNA, 20 bp, Artificial Sequence
  Synthetic oligonucleotide
cgtcccgtagacaaaatggt

SEQ ID NO: 8 - DNA, 20 bp, Artificial Sequence
  Synthetic oligonucleotide
gaatttgccgtgagtggagt

SEQ ID NO: 9 - DNA, 18 bp, Artificial Sequence
  Synthetic oligonucleotide
tcatttgcttcatacagg

SEQ ID NO: 10 - DNA, 18 bp, Artificial Sequence
  Synthetic oligonucleotide
gtgagcaccttccttctt

SEQ ID NO: 11 - DNA, 20 bp, Artificial Sequence
  Synthetic oligonucleotide
ggaatgtgagcaccttcctt

SEQ ID NO: 12 - DNA, 18 bp, Artificial Sequence
  Synthetic oligonucleotide
tcactttcataatgctgg

SEQ ID NO: 13 - DNA, 18 bp, Artificial Sequence
  Synthetic oligonucleotide
aagaaggaaggtgctcac